(19) United States
(12) Patent Application Publication
(10) Pub. No.: US 2017/0290637 A1
(43) Pub. Date: Oct. 12, 2017

(54) SURGICAL SKULL CLAMP

(71) Applicant: CIVAL Medical GmbH, Tuttlingen (DE)

(72) Inventor: Ingolf Diez, Tuttlingen (DE)

(21) Appl. No.: 15/481,408

(22) Filed: Apr. 6, 2017

(30) Foreign Application Priority Data
Apr. 8, 2016 (DE) 102016004231.1

(51) Int. Cl. A61B 90/14; A61B 90/57

(52) U.S. Cl. CPC A61B 90/14; A61B 90/57; A61B 2090/571; A61B 2017/0092

(57) ABSTRACT

The present invention relates to a surgical skull clamp (1) and a method for the manufacture of a surgical skull clamp (1). In one embodiment of the surgical skull clamp (1) at least a part of the surgical skull clamp (1) has at least two different structures.

The present invention relates to a surgical skull clamp and a method for manufacturing a surgical skull clamp.

In (neuro)surgical procedures, the fixation of a skull is mostly unavoidable. To fix the skull, so-called skull clamps (often also described as head clamps or head rests) are used.

Skull clamps of this kind are known, for example, from DE 694 11 621 T2 (=EP 0 623 318 B1) and EP 1 849 427 A1.

Skull clamps available on the market nowadays are manufactured from cast aluminium, for example, or an X-ray transparent material (e.g. carbon). Skull clamps of cast aluminium can be manufactured inexpensively and are relatively stable. However, they are compatible neither with computer tomography (CT) nor with nuclear spin tomography (also termed magnetic resonance tomography (MRT)). Intraoperative radiographs are therefore often not sufficiently precise. In addition, they have a relatively high weight, are prone to wear and are susceptible in processing using cleaning agents.

Although X-ray transparent skull clamps are CT- and MRT-compatible and relatively light, their construction volume is relatively large. Due to the design, the handling of such skull clamps is mostly very laborious and complicated. Such clamps often consist of several components, which cannot be assembled intuitively. The market price of X-ray transparent skull clamps is also relatively high (normally several times that of the aluminium variant).

In most cases, the patient's skull is fixed nowadays in (neuro)surgical procedures with the aid of a skull clamp of cast aluminium.

There is a need, therefore, for improved skull clamps and methods for their manufacture.

According to a first aspect of the invention, a surgical skull clamp is provided. At least a part of the surgical skull clamp has (includes) at least two different structures.

The different structures can lead, for example, to different densities present in the part of the surgical skull clamp. Thus at least the part of the surgical skull clamp can have a first structure and a second structure delimited from the first structure, for example. In the area of the first structure, a first density can be present, i.e. in the area of the first structure at least the part of the surgical skull clamp can have a first density. In the area of the second structure, a second density can be present, i.e. in the area of the second structure at least the part of the surgical skull clamp can have a second density.

Due to the different structures and if applicable the different densities, certain areas can be designed to be more or less stiff. This can have an effect among other things on the construction size. Due to the different structures and if applicable different densities, structures with a small construction volume and low weight, yet high stability, are possible. In addition, a particularly stable material can be used. The material chosen can be compatible with computer tomography (CT) and magnetic resonance (MRT) as well as resistant to cleaning. Furthermore, autoclaving can be made possible. Autoclaving is normally understood as steam sterilisation, thus sterilising in damp heat, in order to free materials or objects from microorganisms in any development stage. Autoclaving normally takes place in the autoclave at temperatures between 110 and 140° C.

The first and second structure, and if applicable the first and second density, can be present in the surgical skull clamp as a whole or just in one part of the surgical skull clamp. At least partially different structures, and if applicable at least partially different densities, can also be present in different parts of the surgical skull clamp. For example, a first and a second structure that is different/divergent/distinct from the first structure can be present in a first part of the surgical skull clamp, a first and a third structure that is different/divergent/distinct from the first and second structure can be present in a second part of the surgical skull clamp, a fourth and a fifth structure can be present in a third part of the surgical skull clamp etc. If the different structure causes a different density, a first and a second density that is different/divergent/distinct from the first density can be present accordingly in a first part of the surgical skull clamp, a first and a third density that is different/divergent/distinct from the first and second density can be present in a second part of the surgical skull clamp, a fourth and a fifth density can be present in a third part of the surgical skull clamp etc.

According to a first possible embodiment, two different structures and if applicable two different densities are present in the surgical skull clamp as a whole. According to a second possible embodiment, two different structures and if applicable two different densities are present in a part of the surgical skull clamp. According to a third possible embodiment, at least two different structures and if applicable two different densities are present in the surgical skull clamp as a whole, wherein the structures and if applicable densities differ at least partially in one or more parts of the surgical skull clamp.

The part of the surgical skull clamp with at least two different structures and if applicable densities can be a component of the skull clamp. The part of the surgical skull clamp with at least two different structures and if applicable densities can be a section of a component or sections of various components.

The surgical skull clamp can have a fixation section for the fixation of a (human) skull. The part with at least two different structures and if applicable densities can be formed as a fixation section or comprise the fixation section. Accordingly at least a part of the fixation section can have at least two different structures and if applicable densities at least in sections.

The surgical skull clamp can have an attachment section for attachment to an operating table. The part with at least two different structures and if applicable densities can be formed as an attachment section or comprise the attachment section. Accordingly at least a part of the attachment section can have at least two different structures and if applicable densities at least in sections.

The surgical skull clamp can have one or more adjusting mechanisms. The adjusting mechanisms are used to adjust the skull clamp. The part with at least two different structures and if applicable densities can comprise at least a part of the one or more adjusting mechanisms or be formed as one or more adjusting mechanisms. Accordingly at least a part of the one or more adjusting mechanisms can have at least two different structures and if applicable densities at least in sections.

A first structure of the at least two different structures can have a first density. A second structure of the at least two different structures can have a second density. The first density can be smaller than the second density or vice versa.

For example, the part of the surgical skull clamp having at least two different structures has a core of a first structure. The core can have a first density. The core can be enclosed by a layer, e.g. external layer, of a second structure deviating from the first structure. The external layer can have a second density distinct from the first density. The external layer can completely enclose the core, for example. The first density can be smaller than the second density, for example. Alternatively the first density can be greater than the second density.

Due to the different structures and if applicable densities, for example due to the core structures, and additionally a suitable material, constructions with a small construction volume and with low weight yet high stability are possible.

At least one part, e.g. component, of the surgical skull clamp can have one or more densities, which change linearly and/or stepwise in one or more directions. For example, the one or more densities can accordingly increase or decrease linearly and/or stepwise. This is regardless of whether the one or more densities are in the external layer, in the core or in another section of the part, e.g. the component.

For example, the at least two different structures (which are present at least in the part of the surgical skull clamp) can have a linearly increasing and/or a linearly decreasing and/or a stepwise increasing and/or a stepwise decreasing and/or a constant density progression at least in sections in a cross sectional direction of the respective part or section of the skull clamp.

At least one of the at least two different structures can lie in an area of the surgical skull clamp that is exposed to above-average stress or loading.

For example, the density can be selected to be very great at certain, e.g. critical, points. This leads to improved stability/stiffness at the certain, e.g. critical, points. Furthermore, on account of the great density, the skull clamp can be configured especially finely at certain areas. Due to the high density it can be achieved that, in spite of the fine configuration, a desired stability/stiffness is still achieved. For example, articulations can be configured very finely. Let it be cited here as a specific example that on account of the fine configuration possibilities, gear rings with a very high number of teeth can be modelled, e.g. 120 teeth instead of, as normal, 48 teeth. This makes substantially finer adjustment possible.

The part of the surgical skull clamp having at least two different structures and if applicable different densities can comprise a honeycomb-like structure and/or a lattice-like structure and/or a pyramid-like structure and/or a sponge-like structure and/or a thread-like structure and/or an open-pored structure and/or a mesh-like structure and/or a spongy structure and/or a textured structure. For example, the core can comprise a honeycomb-like structure and/or a lattice-like structure and/or a pyramid-like structure and/or a sponge-like structure and/or a thread-like structure and/or an open-pored structure and/or a mesh-like structure and/or a spongy structure and/or a textured structure.

The surgical skull clamp can be X-ray transparent, i.e. permeable to X-rays, at least in sections. An X-ray transparent material, for example, can be chosen for this. The surgical skull clamp can consequently be transparent, at least in sections, for computer tomography (CT) procedures. The surgical skull clamp can be transparent, at least in sections, for magnetic resonance tomography (MRT) procedures. It is further possible e.g. in the X-ray transparent variant to use metal indicators also in order to detect the position/location of the skull clamp in the images. According to one embodiment, one or more metal indicators can be arranged on or in the surgical skull clamp.

The surgical skull clamp can be manufactured using or by means of an additive method. The surgical skull clamp can be manufactured from a liquid feedstock, a solid feedstock or a gaseous feedstock. The solid feedstock can comprise a feedstock in the form of wire or stranded feedstock, for example, laminates or a powdered feedstock. In the case of a gaseous feedstock (gaseous initial state of the feedstock), the additive method can comprise thermal evaporation, pulsed laser deposition, cathodic arc deposition or cathode sputtering. In the case of a liquid or paste feedstock (liquid initial state of the feedstock), the additive method can comprise a polymerisation method, in which the liquid or paste feedstock is solidified. In the case of a solid feedstock (solid initial state of the feedstock), the additive method can comprise a powder-processing 3D printing method, laser sintering (also selective laser sintering), mask sintering, beam melting or laser cladding.

According to a second aspect of the invention, a surgical skull clamp is provided. At least a part of the surgical skull clamp has at least two different densities. All of the details described above in relation to the skull clamp according to the first aspect of the invention can be realised in a corresponding manner in the skull clamp according to the second aspect. For example, a first density of the at least two different densities can be caused by a first structure. Furthermore, a second density of the least two different densities can be caused by a second structure deviating from the first structure.

According to a third aspect of the invention, a method for the manufacture of a surgical skull clamp, e.g. the surgical skull clamp described above, is provided. The method comprises a manufacture of at least a part of the surgical skull clamp by an additive method. At least the part of the surgical skull clamp has at least two different structures and if applicable densities.

The method can comprise a provision of a liquid feedstock or of a solid feedstock or gaseous feedstock. The method can further comprise an additive manufacture of the surgical skull clamp from the feedstock provided.

Various materials can be used for the manufacture of the skull clamp. For example, materials can be used that do not lead to any artefacts in the case of an imaging method used. Plastics or metals, for example, can be used as materials. Purely by way of example, polypropylene (PP) and/or polyetheretherketone (PEEK) can be cited here as plastics and titanium, titanium alloys, high-grade steels and aluminium as metals.

With reference to laser sintering, possible specific steps for the manufacture of a skull clamp by means of the additive method are briefly outlined. These steps are not restricted to laser sintering, but can be transferred in a corresponding manner to other additive methods. Laser sintering (also described as selective laser melting) is a generative layering manufacturing process by which the powdered, for example metal and/or ceramic raw materials, can be processed into three-dimensional workpieces of complex shapes, e.g. to the surgical skull clamp described here. To do this, a raw material powder layer is applied e.g. to a substrate and depending on the desired geometry of the workpiece to be produced, is acted upon in a location-selective manner by laser radiation. The laser radiation penetrating into the powder layer causes heating and consequently melting or sintering of the raw material powder particles. Further raw material powder layers are then applied successively to the already laser-treated layer on the substrate until the workpiece has the desired shape and size.

A fourth aspect relates to a computer program with program code means, which, when loaded into a computer or a processor (for example, a microprocessor, microcontroller or digital signal processor (DSP)), or running on a computer or processor (e.g. microprocessor, microcontroller or DSP), causes the computer or processor (e.g. microprocessor, microcontroller or DSP) to execute one or more steps of the method described above. In addition, a program storage medium or computer program product with said computer program is provided. Furthermore, the computer program according to the third aspect, for example, can be stored in a device for manufacturing the skull clamp and cause the device to execute one or more steps of the method.

The computer program can be a control program for controlling the additive method for manufacturing the surgical skull clamp, e.g. for controlling the location-selective action by laser radiation on the raw material powder layer as a function of the desired geometry of the surgical skull clamp.

Even if some of the aspects described above were described in relation to the skull clamp, these aspects can also be realised in a corresponding manner in the method or in the computer program implementing or controlling the method. In just the same way, the aspects described above in relation to the method can be realised in a corresponding manner in the skull clamp.

The present invention is to be explained further with reference to figures. These figures show schematically:

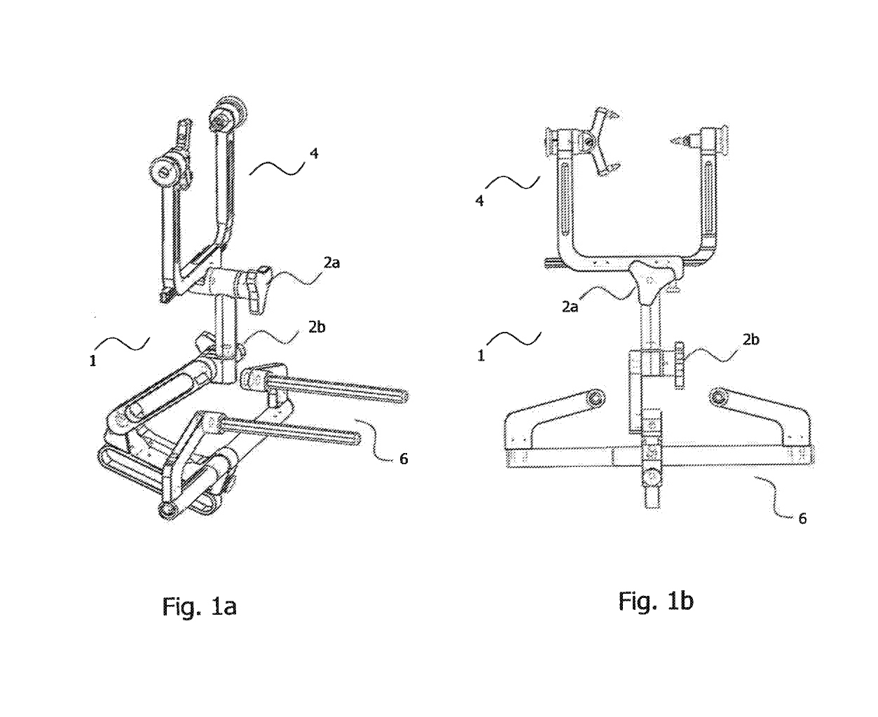

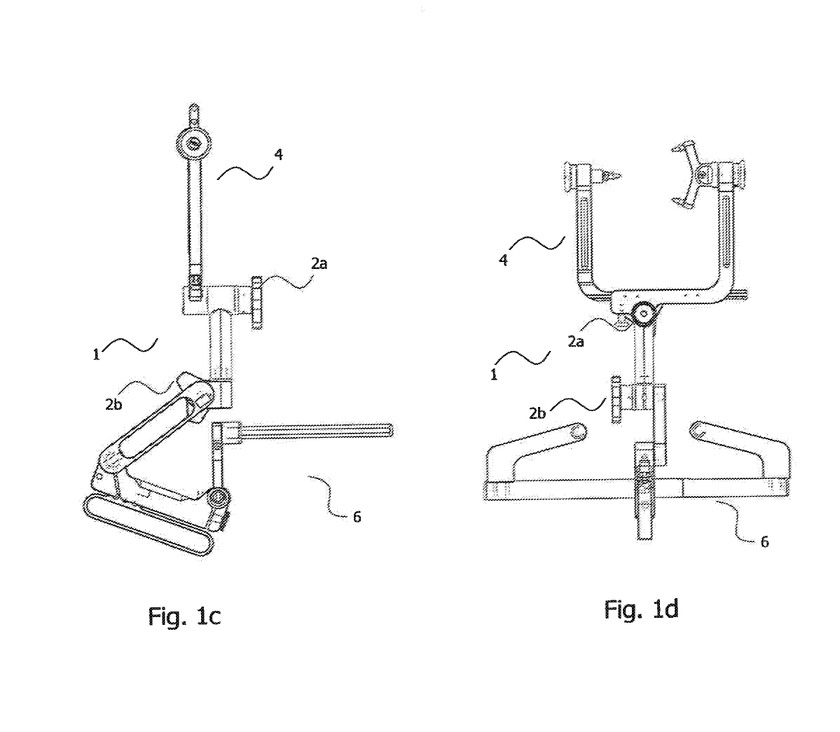

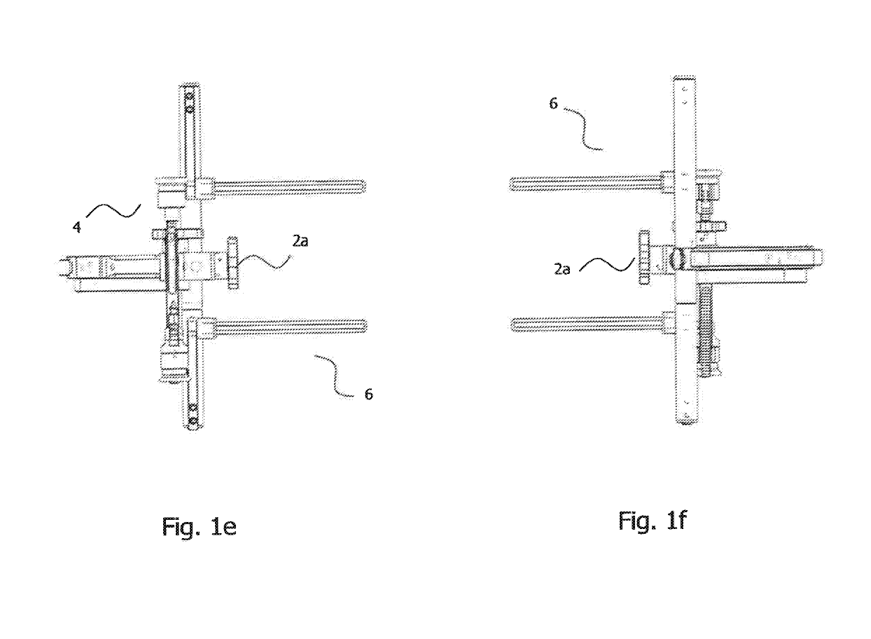

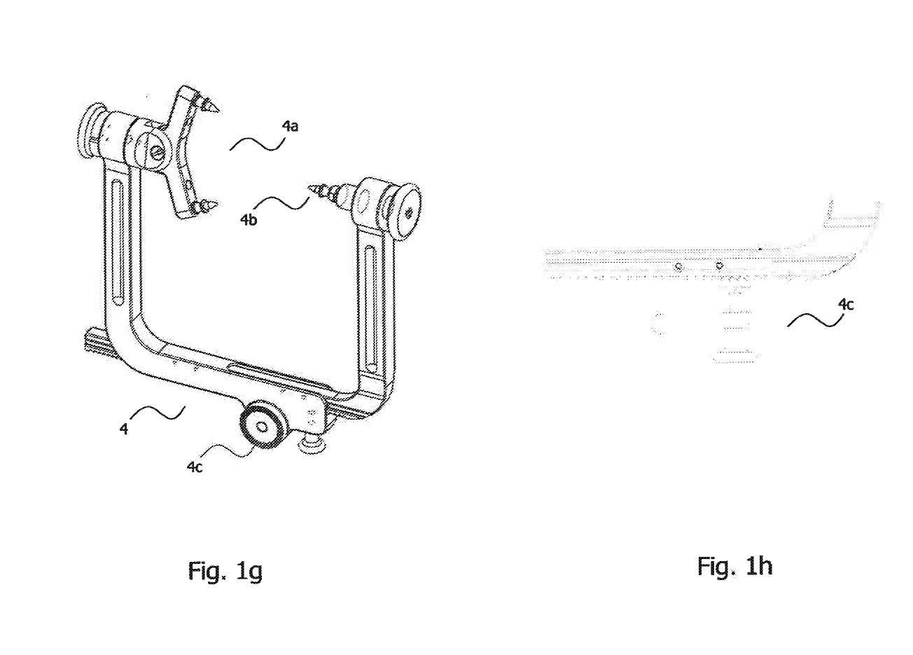

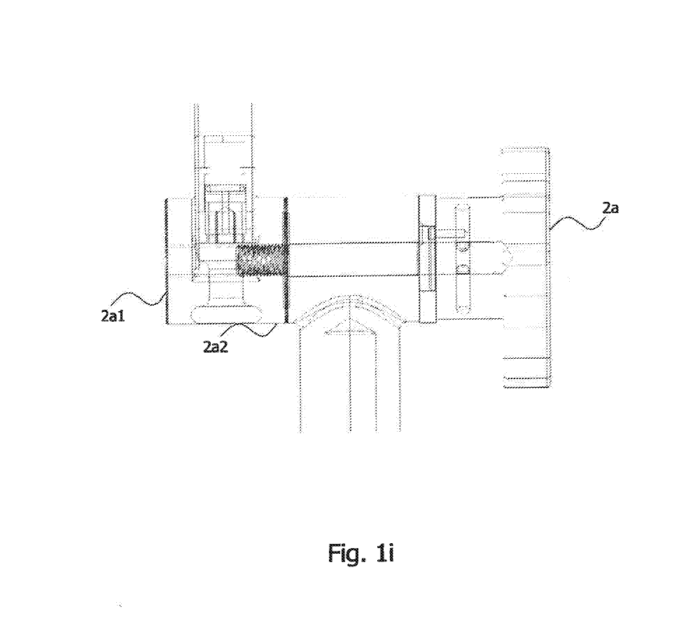

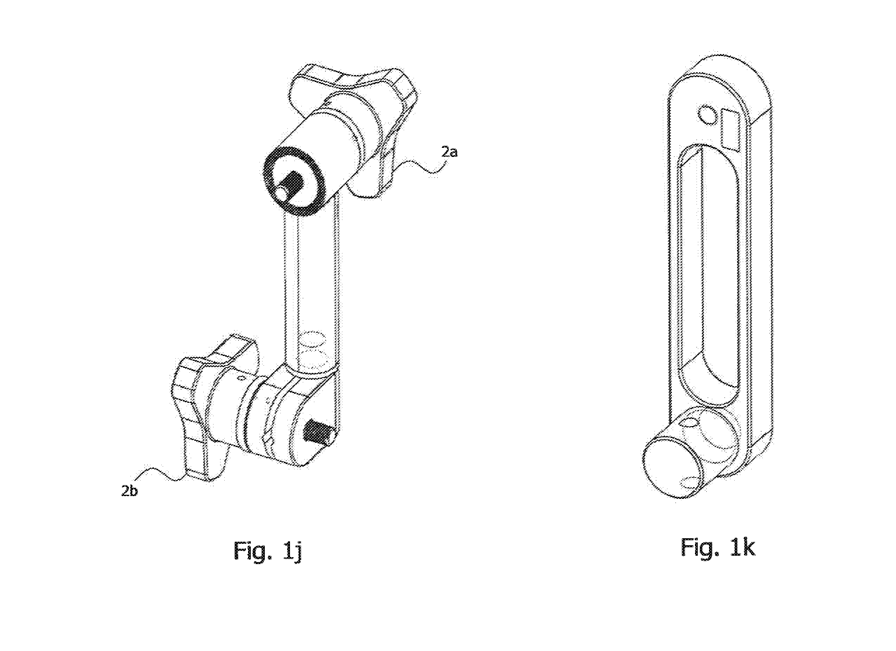

FIGS. 1a to 1m various views of a surgical skull clamp according to an embodiment and details of the surgical skull clamp;

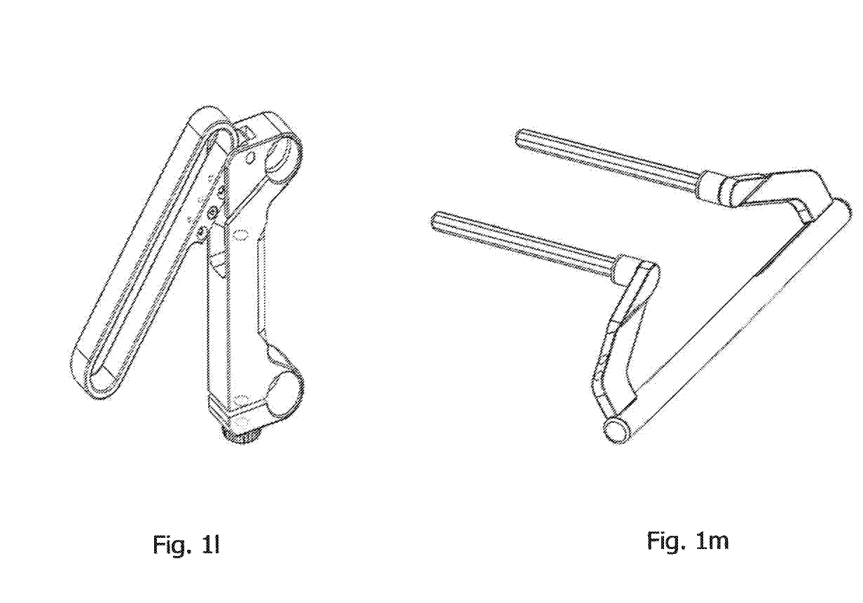

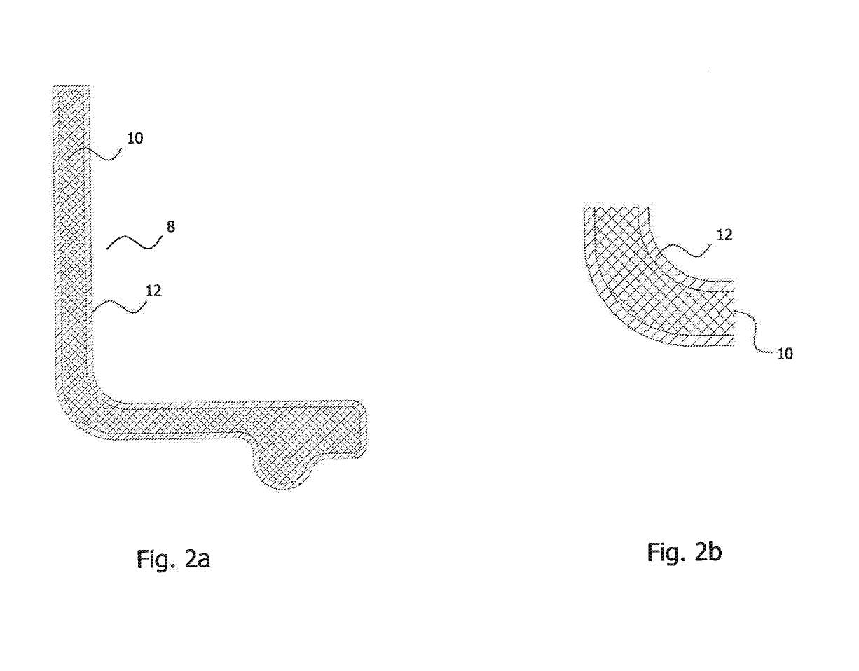

FIGS. 2a and 2b a possible configuration of a part of the surgical skull clamp from FIGS. 1a to 1f;

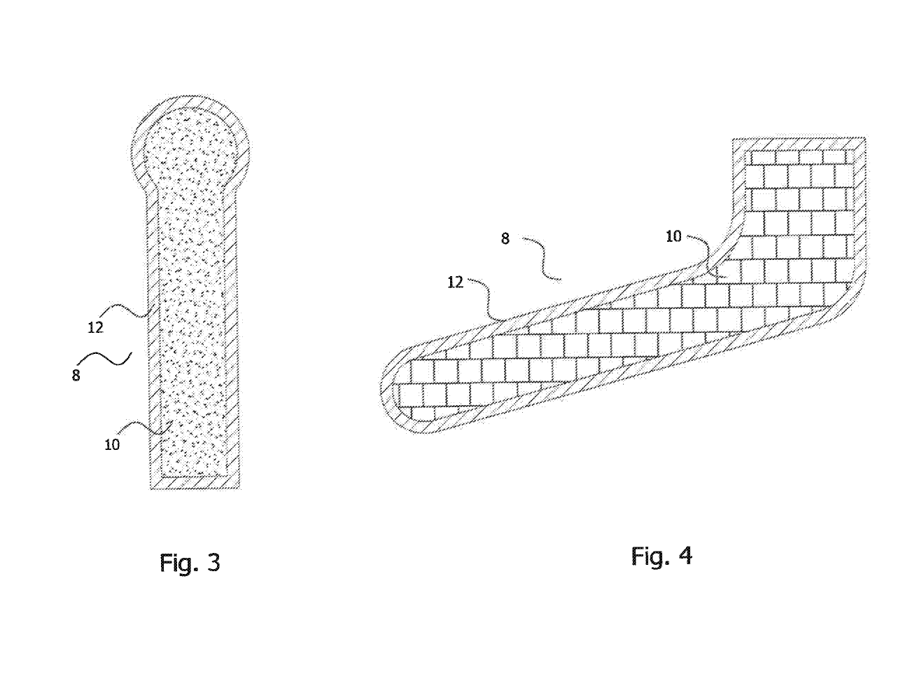

FIG. 3 a possible configuration of a part of the surgical skull clamp from FIGS. 1a to 1f;

FIG. 4 a possible configuration of a part of the surgical skull clamp from FIGS. 1a to 1f;

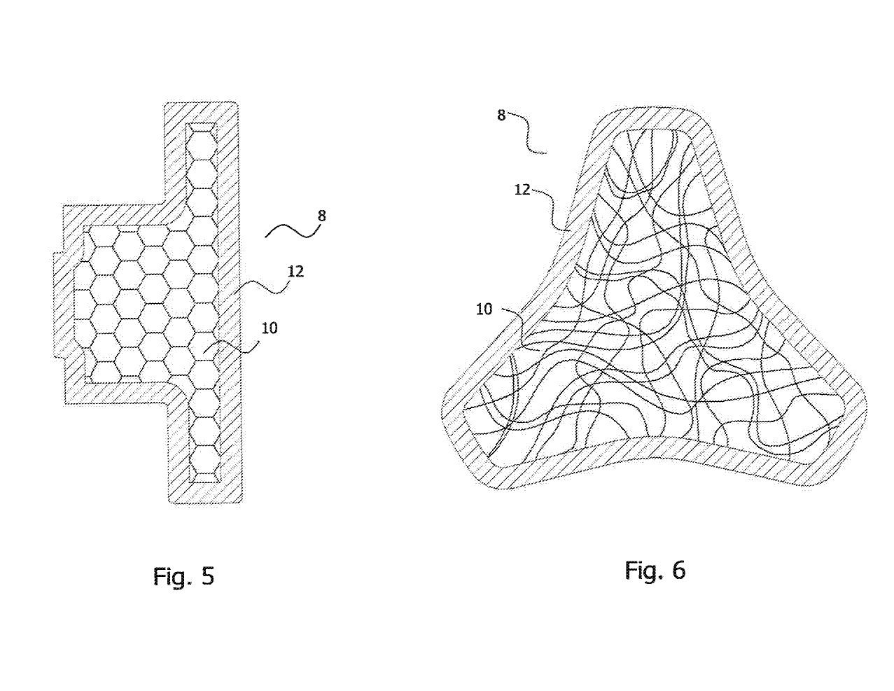

FIG. 5 a possible configuration of a part of the surgical skull clamp from FIGS. 1a to 1f;

FIG. 6 a possible configuration of a part of the surgical skull clamp from FIGS. 1a to 1f;

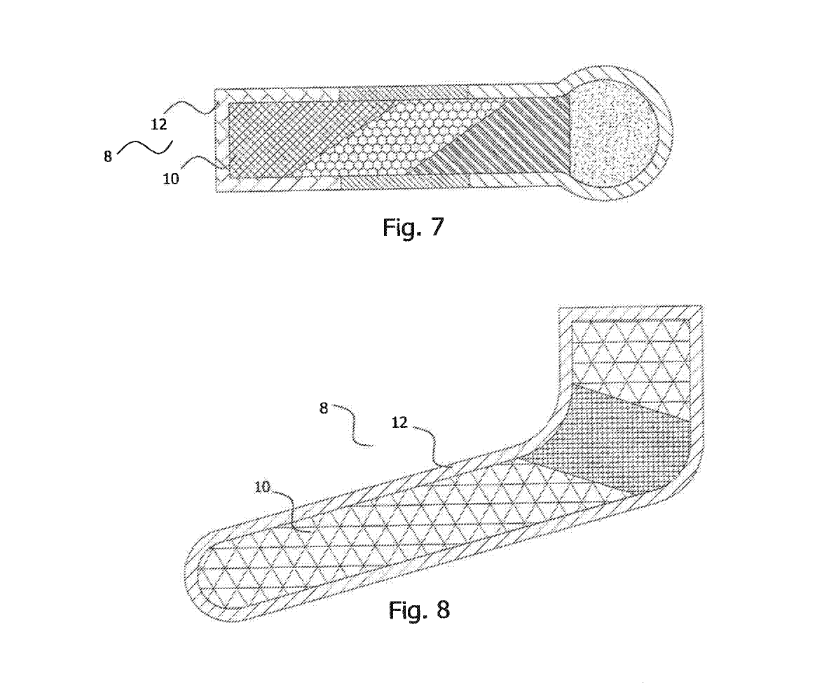

FIG. 7 a possible configuration of a part of the surgical skull clamp from FIGS. 1a to 1f with more than two different structures;

FIG. 8 a possible configuration of a part of the surgical skull clamp from FIGS. 1a to 1f with more than two different structures; and

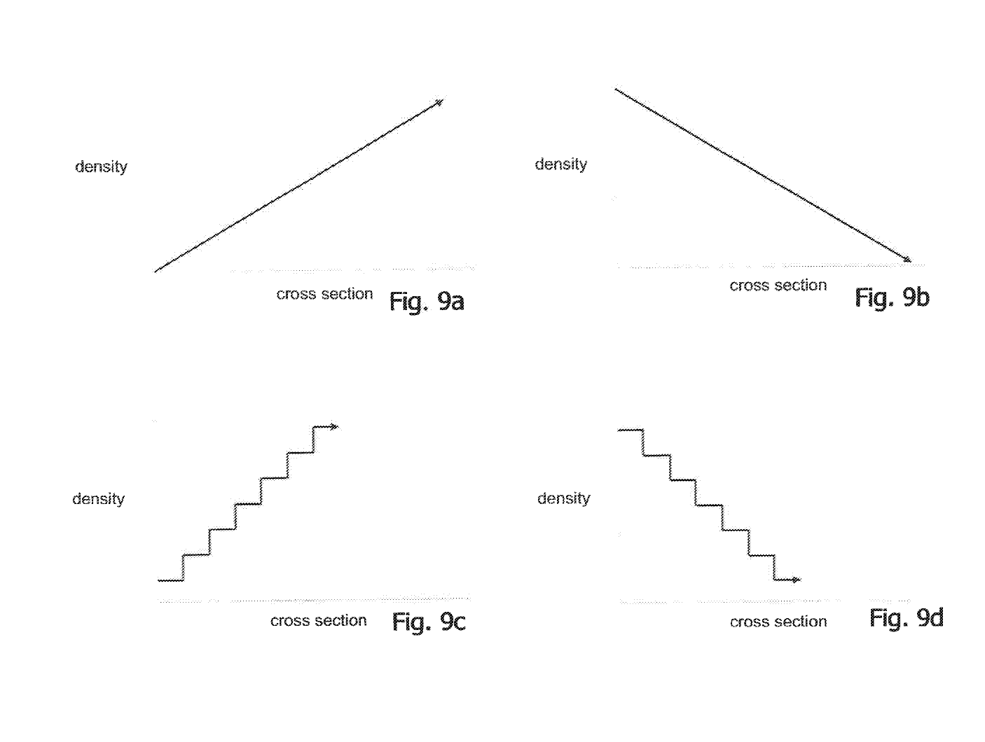

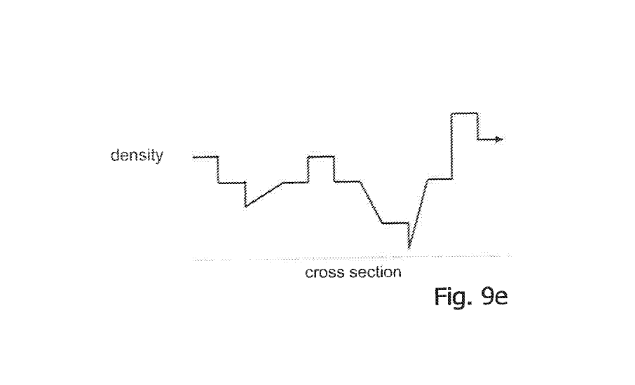

FIGS. 9a to 9e possible density progressions in a part of the surgical skull clamp from FIGS. 1a to 1f.

In the following, without being restricted to these, specific details are presented in order to provide a complete understanding of the present disclosure. However, it is clear to a person skilled in the art that the present disclosure can be used in other embodiments, which can deviate from the details set out below.

FIGS. 1a to 1f show various views of a surgical skull clamp according to an embodiment. FIG. 1a shows an isometric view of the skull clamp 1. FIG. 1b shows a front view of the skull clamp 1. FIG. 1c shows a side view of the skull clamp 1. FIG. 1d shows a rear view of the skull clamp 1. FIG. 1e shows a top view of the skull clamp 1. FIG. 1f shows a bottom view of the skull clamp 1. All representations of the skull clamp are only exemplary, i.e. all elements of the skull clamp 1, such as e.g. handles, articulations etc. can deviate from the representation shown by way of example.

The skull clamp 1 has various sections, components and elements, of which three are mentioned here as an example. As is best recognised in FIG. 1a, the skull clamp 1 has several adjusting mechanisms, of which two are designated below with the reference signs 2a, 2b. If reference is made in the following to the adjusting mechanisms 2a, 2b, a reference to all adjusting mechanisms of the skull clamp can be understood by this, i.e. also to the adjusting mechanisms that are not provided with a reference sign in the figures.

The adjusting mechanisms 2a, 2b are used partly to adjust the skull clamp to individual patient and skull sizes and/or shapes. Thus an exact fixation of a skull is achieved. The skull clamp 1 further has a fixation section 4 for fixation of the skull. Adjusting mechanisms can also be present on the fixation section 4. In addition, the skull clamp 1 has an attachment section 6 for attaching the skull clamp 1 to an operating table (not shown).

In FIGS. 1g to 1m, examples are shown of details and elements of the skull clamp 1. FIG. 1g shows details of the fixation section 4 as an example. In the configuration according to FIG. 1g, the fixation section 4 has a 2-fold fixation 4a and a 1-fold fixation 4b for the fixation of a head. The fixation section 4 also has an adaptation option 4c for accessories. These accessories can comprise a navigation star, retractor systems, a clamp adapter, stereotactic systems and other accessories.

FIG. 1h shows by way of example a latching system of the adaptation option 4c of the skull clamp 1.

FIG. 1i shows a latching fixing of the adjusting mechanism 2a. Gear rings 2a1, 2a2 are provided for adjustment. FIG. 1j shows a rotating head with the adjusting mechanisms 2a, 2b. FIG. 1k shows a support rail of the fixation section 4.

FIG. 1l shows a synchro tensioner, which is provided on the attachment section 6. Finally, FIG. 1m shows a table adapter of the attachment section 6.

At least a part of the skull clamp 1 can have at least two different structures, as described below in relation to FIGS. 2a to 8. Furthermore, the at least two different structures can lead to at least two different densities in the skull clamp 1, as described below in reference to FIGS. 2a to 9e. With reference to FIGS. 2a to 9e, sections of the skull clamp 1 are always spoken of below. However, these sections can also be components or elements of the skull clamp 1. For example, the sections described in relation to FIGS. 2a to 9e can be parts or sections of the elements of the skull clamp 1 shown in FIGS. 1g to 1m or the elements of the skull clamp 1 shown in FIGS. 1g to 1m.

In the following, two different structures and if applicable two different densities are always described with reference to FIGS. 2a and 6. However, each of the sections of the skull clamp 1 shown in FIGS. 2a to 6 can have even more than two different structures and if applicable more than two different densities, as described by way of example in relation to FIGS. 7 and 8. Furthermore, a first structure and if applicable a first density as well as a second structure and if applicable a second density are always spoken of below in relation to FIGS. 2a to 6. These structures and if applicable densities can differ from figure to figure. It is only important that at least two different structures and if applicable two different densities are present in the section illustrated in each figure.

In FIGS. 2a to 8, the section shown in each case is continuously designated by the reference sign 8, in order to illustrate that each section of the skull clamp 1 can be formed correspondingly in principle. The sections from FIGS. 2a to 8 can be one or more of the adjusting mechanisms 2a, 2b, for example, or a part of the one or more adjusting mechanisms 2a, 2b. In addition or alternatively, the sections from FIGS. 2a to 8 can be one or more components of the fixation section 4 or sections of the one or more components of the fixation section 4. In addition or alternatively to this, the sections from FIGS. 2a to 8 can be one or more components of the attachment section 6 or sections of the one or more components of the attachment section 6. In FIGS. 2a to 8, reference is not made below to a specific component of the skull clamp. The details presented below are applicable accordingly to all sections, parts, components or elements of the skull clamp 1. In one embodiment, the entire surgical skull clamp consists of two or more structures and if applicable of two or more densities, i.e. the entire surgical skull clamp is constructed as described in relation to FIGS. 2a to 8. In connection with the last-named embodiment, it is important that different structures, such as described by way of example in relation to FIGS. 2a to 8, can be combined with one another in the skull clamp. The densities of the different structures described below can differ from one another at least partially.

As is to be recognised in FIGS. 2a and 2b, the section 8 having two different structures has a core 10 and an outer layer (external layer) 12 surrounding the core 10. The core 10 is shown with a lattice structure as an example in FIG. 2a. The core 10 has a first density. The outer layer (external layer) 12 has a different type of structure. The outer layer 12 has a second density. The lattice-like structure of the core 10 gives the core a first density distinct from the second density of the external layer 12. This means that the core 10 has a first density, which is different from the density of the outer layer 12.

According to FIGS. 2a and 2b, the section 8 having the two different structures consists of a core 10 and an outer layer (external layer) 12 surrounding the core 10. No other layers or structures are present. Accordingly the section 8 in the configuration shown as an example has no density deviating from the densities of the core 10 and the external layer 12.

FIGS. 3 to 6 illustrate schematically that other structures are conceivable for the core 10. For the external layer 12, the same structure is always assumed by way of example for each of the sections 8. However, even the structure of the external layer 12 of the sections 8 can vary.

The respective structures of the core 10 give the core 10 a density deviating from the first density of the outer layer 12. As an example, a structure of the core 10 is to be recognised in FIG. 3 that can be described as a spongy/sponge-like structure. The density of the core 10 with the spongy/sponge-like structure differs from the density of the external layer 12.

As another example, a lattice-like or wall-like structure is shown for the core 10 in FIG. 4. The density of the core 10 with a lattice-like or wall-like structure also differs from the density of the external layer 12.

As another example, a honeycomb-like structure for the core 10 is shown in FIG. 5. The density of the core 10 with a honeycomb-like structure also differs from the density of the external layer 12.

In FIG. 6 a thread-like structure is shown for the core 10. The density of the core 10 with a thread-like structure also differs from the density of the external layer 12.

All of the examples represented in FIGS. 2a to 6 illustrate that a density for the core 10 deviating from the outer layer 12 can be achieved in a different way, i.e. with different types of structures. As an example, the core 10 according to the embodiments from FIGS. 2a to 6 can have a lower density than the density of the outer layer 12. Alternatively, the core 10 according to the embodiments from FIGS. 2a to 6 can have a higher density than the density of the outer layer 12.

FIG. 7 illustrates schematically that more than two other structures are conceivable for the core 10 and the external layer 12. Purely by way of example, the section 8 from FIG. 7 has three different structures in the external layer 12. These three different structures lead to three different densities in the external layer 12. Purely by way of example, the section 8 from FIG. 7 has four different structures in the core 10. These four different structures lead to four different densities in the core 10.

FIG. 8 illustrates schematically how a certain area (here: a critical area) of the section 8 can be provided with a structure other than other areas of the section 8.

Purely by way of example and without being restricted to this, this section is a curved section in the core 10 of the section 8. This curved section in the core 10 has a structure with a higher density than the other structures of the core 10 and the external layer 12. This increases the stability/stiffness in the curved and thus critical section.

In FIGS. 2a to 8, the section 8 was described in each case by way of example in such a way that it has a core and an external layer, e.g. consists of a core and an external layer. However, the realisations from FIGS. 2a to 8 are not restricted to this, as is illustrated as an example in relation to the density progressions shown schematically in FIGS. 9a to 9e.

FIGS. 9a to 9e show possible progressions of the density in the respective sections of the skull clamp 1 or in the skull clamp 1 as a whole from FIGS. 1a to 1f.

It is possible, for example, that the density increases linearly along a cross section through a corresponding section 8, as shown in FIG. 9a. It is also possible that the density decreases linearly along a cross section through a corresponding section 8, as is shown in FIG. 9b. The density progressions of FIGS. 9a and 9b can also be combined. The density can accordingly increase linearly initially along a cross section through a corresponding section 8, according to FIG. 9a, before then, according to FIG. 9b, decreasing again linearly along a cross section through a corresponding section 8. Conversely, the density can decrease linearly initially along a cross section through a corresponding section 8, according to FIG. 9b, before then, according to FIG. 9a, increasing again linearly along a cross section through a corresponding section 8.

It is further possible that the density increases stepwise along a cross section through a corresponding section 8, as shown in FIG. 9c. It is also possible for the density to decrease stepwise along a cross section through a corresponding section 8, as shown in FIG. 9d. The density progressions of FIGS. 9c and 9d can also be combined. The density can accordingly increase stepwise initially along a cross section through a corresponding section 8, according to FIG. 9c, before then, according to FIG. 9d, decreasing again stepwise along a cross section through a corresponding section 8. Conversely, the density can decrease stepwise initially along a cross section through a corresponding section 8, according to FIG. 9d, before then, according to FIG. 9c, increasing again stepwise along a cross section through a corresponding section 8.

The density progressions of FIGS. 9a to 9d can be combined with one another in any way. Even non-rising/non-falling, i.e. constant density progressions are possible and can be combined in any way with the progressions from FIGS. 9a to 9d. Thus FIG. 9e shows purely by way of example a density profile along a cross section of the section 8.

1-12. (canceled)

13. Surgical skull clamp, wherein at least a part of the surgical skull clamp has at least two different structures.

14. Surgical skull clamp according to claim 1, wherein the surgical skull clamp has:
a fixation section for the fixation of a skull; and/or
an attachment section for attachment to an operating table; and/or
one or more adjusting mechanisms;
wherein at least a part of the fixation section and/or a part of the attachment section and/or a part of the one or more adjusting mechanisms have at least two different structures at least in sections.

15. Surgical skull clamp according to claim 1, wherein a first structure of the at least two different structures has a first density and a second structure of the at least two different structures has a second density, wherein the first density is smaller than the second density or vice versa.

16. Surgical skull clamp according to claim 1, wherein the part of the surgical skull clamp has a core of a first structure, which is enclosed by a layer of a second structure.

17. Surgical skull clamp according to claim 1, wherein the part of the surgical skull clamp comprises: a honeycomb-like structure and/or a lattice-like structure and/or a pyramid-like structure and/or a sponge-like structure and/or a threadlike structure and/or an open-pored structure and/or a mesh-like structure and/or a spongy structure and/or a textured structure.

18. Surgical skull clamp according to claim 1, wherein the surgical skull clamp is completely or partially X-ray transparent, at least in sections.

19. Surgical skull clamp according to claim 1, wherein the surgical skull clamp is manufactured using an additive method.

20. Surgical skull clamp according to claim 1, wherein the at least two different structures have a linearly increasing and/or a linearly decreasing and/or a stepwise increasing and/or a stepwise decreasing and/or a constant density progression at least in sections in a cross sectional direction.

21. Surgical skull clamp according to claim 1, wherein at least one of the at least two different structures lies in an area of the surgical skull clamp that is exposed to above-average stress or loading.

22. Surgical skull clamp according to claim 1, wherein one or more metal indicators are arranged on or in the surgical skull clamp.

23. Surgical skull clamp, wherein at least a part of the surgical skull clamp has at least two different densities.

24. Method for the manufacture of a surgical skull clamp, wherein the method comprises: manufacture of at least a part of the surgical skull clamp using an additive method, wherein the part of the surgical skull clamp has at least two different structures.
